(19) United States
(12) Patent Application Publication
(10) Pub. No.: US 2011/0071356 A1
(43) Pub. Date: Mar. 24, 2011

(54) Repeatably flexible surgical instrument

(75) Inventor: Kevin C. Edwards, Olive Branch, MS (US)

(73) Assignee: GYRUS ENT, L.L.C., Bartlett, TN (US)

(21) Appl. No.: 12/585,788

(22) Filed: Sep. 24, 2009

(51) Int. Cl. A61B 1/008

(52) U.S. Cl. 600/142

(57) ABSTRACT

A flexible-shaft surgical instrument is provided that can be repeatably bent to one or more desired angles on demand by a user without the use of a special bending tool. The flexible-shaft surgical instrument can be repeatably bent and re-bent into multiple positions without plastic deformation of the flexible-shaft surgical instrument occurring in any of one or more bend portions.

### BACKGROUND

This disclosure relates to an improved surgical instrument structure for surgical instruments such as shavers and microdebriders.

Surgical instruments with thin, elongated shafts for accessing various surgical sites through natural openings in the body, or through surgically-inserted cannulae, are known. These surgical instruments may be provided with generally thin, elongated shafts in either straight or curved configurations. An illustrative example of a curved shaft surgical instrument is the curved-shaft shaver blade surgical instrument disclosed in U.S. Pat. No. 4,646,738 to Trott.

In thin elongated-shaft surgical instruments, it is often useful, or even necessary, for a surgeon to be able to precisely orient the instruments' distal end with respect to the target site in the patient's body during surgery. This is a relatively straightforward procedure with straight-shaft surgical instruments. This procedure, however, tends to become more complicated with curved-shaft surgical instruments. An example of a curved-shaft surgical instrument is the curved-shaft shaver blade surgical instrument, specifically adapted so that the cutting window located proximally adjacent its distal end can be reasonably easily re-oriented, is provided in U.S. Pat. No. 5,411,514 to Fucci et al.

Variations to the rigid straight-shaft or curved-shaft surgical instruments, such as the shaver blades discussed briefly above, have been introduced. Flexible-shaft surgical instruments have been developed. In such devices, generally, a powered hand piece drives a flexible inner surgical member, such as an inner cutting member in a flexible-shaft shaver blade surgical instrument. The flexible inner surgical member is generally housed and supported in a semi-rigid outer elongated thin shaft. The semi-rigid outer elongated thin shaft generally provided with at least one bendable portion. The at least one bendable portion differentiates conventional flexible-shaft surgical instruments from conventional rigid curved-shaft surgical instruments. Conventional flexible-shaft surgical instruments often include outer shafts members in which the bendable portion is a single continuous tube having, for example, a ribbed portion with alternating thick and thin wall thicknesses along the length of the shaft in a specific bend region. This allows for the outer shaft to be bent more easily than a non-ribbed conventional outer shaft member with a constant wall thickness in its bend region. Such construction is intended to reduce, for example, crimping in the bendable portion that could impact the inner flexible surgical member rendering it partially or wholly inoperable.

A number of difficulties have been encountered in attempting to develop reliable flexible-shaft surgical instruments, particularly in developing such instruments that may be repeatedly bendable. To reduce the frequency of a possibility of crimping, for example, some flexible-shaft surgical instruments require a compatible bending tool to provide a user with a controlled method for bending the device to a desired angle according to a specific bend radius. Without the use of such a bending tool, the flexible shafts of the devices are subject to the above-mentioned crimping through user error resulting in the outer shafts of the surgical instruments being bent, for example, at too great an angle or according to too small a bend radius.

Other problems with conventional flexible-shaft surgical instruments include that, even if not crimped, they are often subject to some plastic deformation, once bent, leading to limited reusability. If bends are not controlled, or bends are made too often, the tubes of the conventional flexible-shaft surgical instrument may kink, crimp, collapse, rupture or otherwise fail. This plastic deformation may also negatively affect the function of the inner surgical instrument such as the cutting member on a first or subsequent uses.

An additional drawback to a conventional flexible-shaft surgical instrument is that the plastic deformation that occurs in any of the tubes prevents the device from being bent in different planes successfully and successively. Once a tube is bent one time in a conventional flexible-shaft surgical instrument, it may never regain its original shape. Therefore, conventional flexible-shaft surgical instruments are limited to a finite set of orientations, e.g. concave, convex, left, right, and bend angle combinations, as well as minimal, if any, re-use.

### SUMMARY

User preferences, such as those of surgeons, modified by their patients' needs, dictate limitless combinations of bend parameters with regard, for example, to angles of the bend and window positions, in the case of flexible-shaft surgical instruments. Accommodating such user preferences, and the full scope of patient anatomies, during surgical procedures is often difficult with the use of conventional flexible-shaft surgical instruments. Ease of bending a flexible-shaft surgical instrument, as well as enabling a surgeon to adapt on the fly in a surgery, is also difficult with a conventional flexible-shaft surgical instrument, particularly one that plastically deforms when bent.

Additional restrictions on flexible-shaft surgical instruments include that they are required to be formed of materials that are biocompatible. Further, if the flexible-shaft surgical instruments are to be re-used, the materials from which they are formed need to be able to withstand the rigors of repeated cleaning and sterilization.

It would be advantageous to provide a flexible-shaft surgical instrument that can be bent to a desired angle, or combination of desired angles in more than one plane, on demand by a user. To any extent that such flexible-shaft surgical instruments can be bent without the use of a special bending tool, provides an additional advantage to the user. Further, a flexible-shaft surgical instrument that can be repeatedly bent and re-bent into multiple positions without plastic deformation of the flexible-shaft surgical instrument occurring in any of its bend portions is also highly desirable.

Considerations in the design and development of each surgical instrument include the following. It is preferable that the surgical instrument be made of biocompatible materials. It is further preferable that the surgical instrument can be sterilized after each use without affecting necessary compatibilities or bending capacity, including a capacity to be bent along multiple axes singly, or in a compound manner. The materials must be assembled such that the device is able to be easily deformed yet retain its deformed shape, once manipulated, without returning to a pre-deformed shape. In other words, though flexible, the shaft of the instrument must be sufficiently rigid in its bend portion such that, once deformed, the flexible shaft of the surgical instrument will retain its deformed shape according to the user's formation of the shaft. The shaft must be formed from wear resistant and heat resistant materials. The materials are preferably easily manufactured and, preferably, non-conductive. The materials should be selected to avoid other problems with particular materials such as brittleness, lack of flexibility and poor machinability.

It would be advantageous to provide a flexible-shaft surgical instrument capable of being easily manipulated by surgeon control without the traditional drawbacks of a conventional flexible-shaft surgical instrument, discussed above. Such a device could operate as a “one-size-fits-most” single-device solution to using multiple devices, and could allow customization of the bend parameters for the surgeon user.

It would be advantageous to provide a flexible-shaft surgical instrument that may be repeatably bendable in a manner particularly where the bend portion is wear resistant to prevent degradation of a semi-rigid bend portion and enable re-use of the device. The flexible-shaft surgical instrument may be formed of a creep-resistant material such that the deformation characteristics of the device are not negatively affected over the course of its shelf life. The flexible shaft surgical instrument may be formed of a material that is additionally temperature resistant so that the flexible-shaft surgical instrument may survive multiple sterilization procedures to allow the flexible-shaft surgical instrument to be used multiple times. The flexible-shaft surgical instrument may be formed from a material that is non-conductive to prevent electrical shorts between the flexible-shaft surgical instrument, and other surgical instruments that are used in cooperation with the flexible-shaft surgical instrument. The flexible-shaft surgical instrument may also be formed of a material that is non-reactive with most chemicals encountered in a surgical procedure in which the flexible-shaft surgical instrument may be used.

In various exemplary embodiments, a flexible-shaft surgical instrument may be provided that includes at least one semi-rigid shaft portion. The at least one semi-rigid shaft portion may advantageously incorporate a ball-and-socket link structure to make a flexible, but rigid, tube portion in which a plurality of modular segments may be snapped into place to form a repeatedly bendable structure. These modular segments may enable a surgeon to control the degree of bend of the semi-rigid shaft portion and enable the surgeon to return the instrument to a straight orientation to be later re-bent and re-used without plastic deformation.

Devices according to this disclosure may include surgical instruments having repeatably flexible outer shaft portions in order to accommodate a wide array of internal surgical components. Devices according to this disclosure may provide surgical instruments having outer portions that have an opening at or near a distal end such as a cutting window or a portal to otherwise accommodate any manner of surgical instrument. These surgical instruments include, but are not limited to, a surgical light, camera or other observation device, or some manner of surgical instrument tip for cutting, cauterizing or otherwise treating a surgical site within a patient. A cutting window, in a shaver blade instrument, for example, within an outer portion may allow for engagement between a patient's tissue and a cutting element in the inner portion.

In various exemplary embodiments, the flexible-shaft surgical instrument should not require the use of a bending tool to make a “correct” bend. The flexible-shaft surgical instrument may be bendable by a multitude of techniques and according to surgeon user preferences. The flexible-shaft surgical instrument may also be bent without plastic deformation to the bendable portion that takes place when the flexible-shaft surgical instrument is bent. Therefore, a semi-rigid shaft of the flexible-shaft surgical instrument may be bent multiple times in multiple planes or may otherwise be returned to a substantially straight orientation after bending. The reliability of such a device is not compromised when the semi-rigid shaft is bent, and its life expectancy may be increased when compared to conventional flexible-shaft surgical instruments.

In various exemplary embodiments, the device may be scalable, allowing flexible-shaft surgical instruments to be assembled which may be directed to differing patient anatomies (such as children and adult sizes), and to specific operating procedural requirements (e.g., nasopharyngeal or sinus procedures).

In various exemplary embodiments, the device may also have electrosurgical components added to the flexible-shaft surgical instrument using similar assembly materials and methods existing in known flexible-shaft surgical instruments.

In various exemplary embodiments, the flexible-shaft surgical instrument may include ball-and-socket links, described in paragraph [0014] above, that are machined, molded or otherwise formed from wear-resistant and biocompatible polymer compounds specifically adapted to such use, such as polyetherimide.

In various exemplary embodiments, the flexible-shaft surgical instrument may also have one or more rigid shaft members that extend from either side of the at least one semi-rigid portion, and also may incorporate a protective sheath to protect at least one semi-rigid portion, as well as any rigid portions of the flexible-shaft surgical instrument.

In various exemplary embodiments, the flexible-shaft surgical instrument may be a flexible shaver blade having a cutting window and blade portion for cutting a patient's tissue. However, the flexible-shaft surgical instrument may take other forms such as an endoscope, light, camera, vacuum, suction lumen, electrosurgical instrument and the like.

These and other features and advantages of the disclosed device are described in, or apparent from, the following detailed description of various exemplary embodiments.

### BRIEF DESCRIPTION OF THE DRAWINGS

Various exemplary embodiments of the disclosed flexible-shaft surgical instrument will be described, in detail, with reference to the following drawings wherein:

FIG. 1 illustrates a perspective view of an exemplary flexible-shaft surgical instrument that incorporates a semi-rigid portion according to the disclosure;

FIG. 2 illustrates a perspective view of an exemplary semi-rigid portion of a flexible-shaft surgical instrument including a series of ball-and-socket links according to the disclosure;

FIG. 3 illustrates a perspective view of an exemplary flexible-shaft surgical instrument that is a flexible shaver blade having a protective sheath according to this disclosure;

FIG. 4 illustrates a perspective view of an exemplary flexible-shaft surgical instrument that is a flexible shaver blade illustrating ball-and-socket links as the semi-rigid portion;

FIG. 5 illustrates a perspective view of a second exemplary flexible-shaft surgical instrument including a series of ball-and-socket links as its semi-rigid portion that vary in size and that are interchangeable; and

FIG. 6 illustrates a perspective view of a third exemplary flexible-shaft surgical instrument that incorporates two semi-rigid portions.

### DETAILED DESCRIPTION OF EMBODIMENTS

The following embodiments illustrate examples of a flexible-shaft surgical instrument that may be bent to a desired angle on demand by a user without the use of a special bending tool. Disclosed embodiments of the flexible-shaft surgical instrument may be repeatably bent and re-bent into multiple positions without plastic deformation of the flexible-shaft surgical instrument occurring in any of its bend portions. While the disclosed embodiments may refer specifically to a repeatably bendable surgical instrument such as a shaver blade surgical instrument, this example is provided only as being illustrative of a surgical instrument which may gain special advantage based on the repeatably bendable configuration of a semi-rigid shaft portion according to this disclosure. It should be recognized, however, that a device including a semi-rigid shaft portion according to this disclosure may find utility in supporting any manner of surgical instrument where, for example, access is gained to a target surgical site inside a patient's body via one or more natural openings in the patient's body and/or via one or more surgically-inserted cannulae. In this regard, specific disclosed examples of surgical instruments, and the use of specific terms to describe those instruments, should be considered as illustrative only, and not limiting.

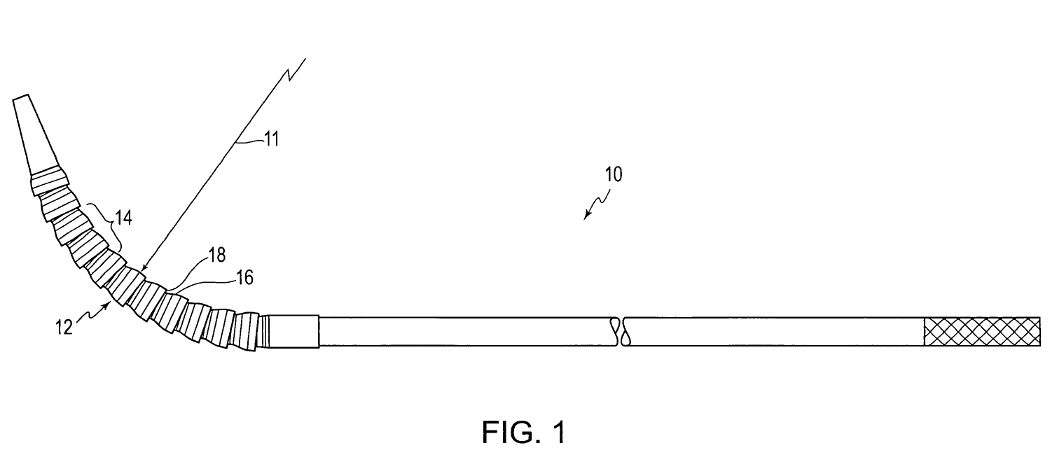

FIG. 1 illustrates a perspective view of an exemplary flexible-shaft surgical instrument 10 that incorporates a semi-rigid portion 12. The semi-rigid portion 12 in this exemplary embodiment comprises a plurality of ball-and-socket links 14 that form a repeatably bendable structure. Each of the ball-and-socket links 14 may be formed from a biocompatible polymer material such as polyetherimide. Each ball-and-socket link 14 may comprise a male portion 16 inserted into a female portion 18 of an adjacent ball-and-socket link 14. The spherical diameter of the male portion 16 may be slightly larger than the spherical diameter of an adjacent female portion 18 prior to assembly. This size difference allows for deflection in the walls of the female portion 18 as the ball-and-socket links 14 are snapped together. This configuration is intended to create a press fit between the mated surfaces of the male portion 16 and the adjoining female portion 18 of adjacent ball-and-socket links 14 in order that the bend portion retains its bend once bent. The unassembled difference in diameters between the male portion 16 and adjacent female portion 18 may advantageously be approximately 3-5%.

The above-described size relationship between portions of adjacent components facilitates in maintaining the stiffness between adjacent links, and therefore the reliability of the form of the semi-rigid portion, without negatively affecting the repeatably bendable reliability of the device. If the difference between the spherical diameter of the male portion 16 and the spherical diameter of the adjacent female portion 18 is too small, the semi-rigid portion 12 of the flexible-shaft surgical instrument 10 will not hold its set while in use. If the difference is too large between the male portion 16 and adjacent female portion 18, the semi-rigid portion 12 may fracture when assembling the links, or may otherwise be rendered too stiff to be bent upon the application of reasonable force in use. The flexible-shaft surgical instrument 10 may have adjacent male 16 and female 18 portions of the modular ball-and-socket links that have spherical diameters on the order of 5-6 mm.

Flexible-shaft surgical instruments traditionally have a center line radius 11 which determines the extent that the semi-rigid portion 12 may be bent. Depending on the number and size of the ball-and-socket links 14, the center line radius 11 can range from zero (in the case where there is only one ball-and-socket link 14) to two inches. The semi-rigid portion 12, again depending on the number and configuration of the ball-and-socket links 14, may be bendable between angles ranging from 0° to 275°. The semi-rigid portion 12 having at least three adjacent links 14 may also be bendable in different planes, at the same time, or successively. Various arrangements of the ball-and-socket links 14 of the flexible-shaft surgical instrument may have varying holding strengths between particular ball-and-socket links 14.

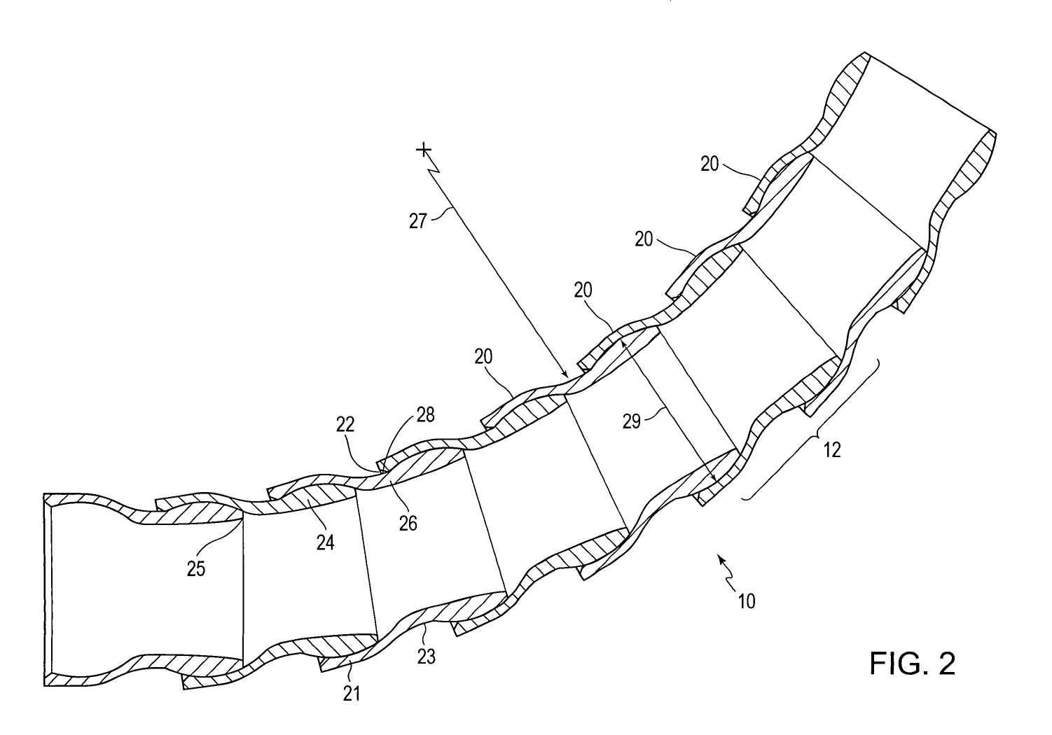

FIG. 2 illustrates a perspective view of a series of ball-and-socket links 20 that make up a semi-rigid portion 12 of a flexible-shaft surgical instrument 10. An entry region 28 of a female portion 22 of a ball-and-socket link 20 is larger than the adjacent male portion 24 of a ball-and-socket link 20 to facilitate assembly. An inner portion of the female portion 22 is smaller than the adjacent male portion 24 so that the male portion 24 may pop into position when assembled.

The amount of surface area that is shared between the male portion 24 and adjacent female portions 22 of the ball-and-socket links 20 may affect the general stiffness of the links that make up the semi-rigid portion 12 of the flexible-shaft surgical instrument 10. The thickness 21 of the ball-and-socket links 22 and a flat portion 23 of the ball-and-socket links 22 should preferably be not less than 5% of the overall width of the ball-and-socket link 22. If the thickness 21 or flat region 23 is less than 5%, the ball-and-socket link 20 may be susceptible to fracture during assembly.

A radius of curvature 25 of the male portion 24 of the ball-and-socket link 20 approximates the desired center line radius 27 of the flexible-shaft surgical instrument 10. This allows the adjacent female portion's inner diameter 29 to remain constant whether it is in a straight or bent orientation. Otherwise, the inside edge 25 at the extreme male end 24 of the semi-rigid portion 12 may protrude into the inner diameter of the device in one axis of its cross section when bent. This protrusion may squeeze a surgical device that is inside of the flexible-shaft surgical instrument, such as a flexible shaver, light or camera, and negatively affect the performance of the device.

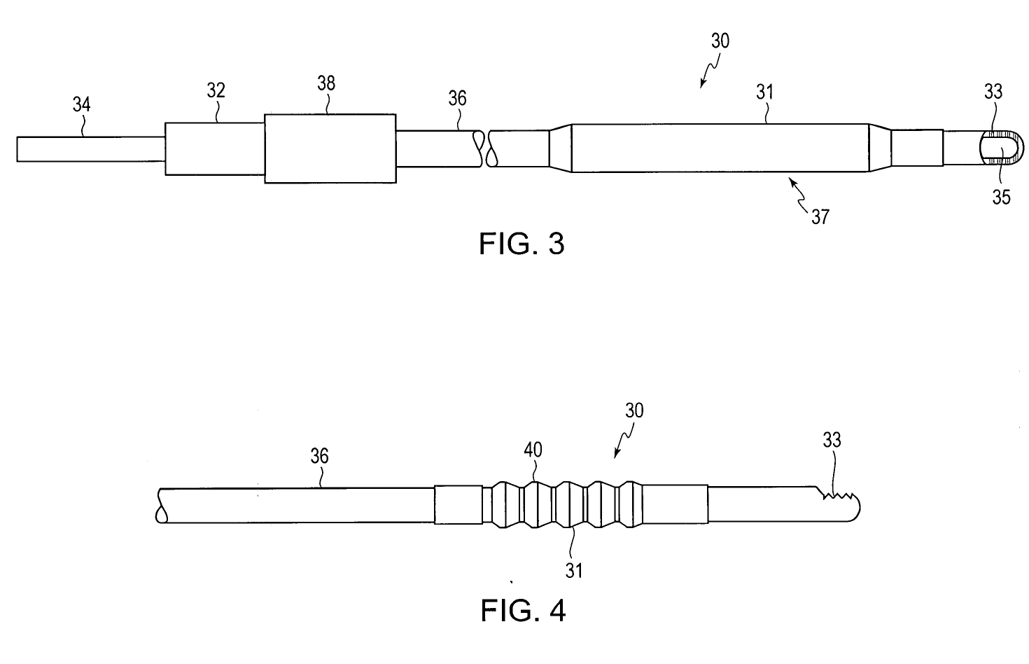

FIG. 3 illustrates a perspective view of a flexible-shaft surgical instrument that is a flexible shaver blade 30 that comprises an inner tube 34 that has a proximal end and a distal end. The inner tube 34 has a cutting element adjacent to its distal end. The flexible-shaft surgical instrument further comprises a first hub 32 that is connected to the proximal end of the inner tube 34 and allows the inner tube 34 to be driven for rotation along a central axis. The flexible shaver blade 30 further comprises an outer tube 36 that has a second hub 38. The inside diameter of the outer tube 36 is larger than an outside diameter of the inner tube 34. The outer tube 36 comprises a proximal portion attached to the second hub 38 and a semi-rigid portion 31 that has a proximal end that is attached to the proximal portion of the outer tube 36. The outer tube 36 has a distal portion that is attached to a distal end of the semi-rigid portion 31 and provides for a cutting window 35 at, or in the vicinity of, the extreme distal end of the instrument. The distal portion of the outer tube 36 provides for the cutting window 35 to face a predetermined direction and enables the cutting element 33 to cut tissue. The semi-rigid portion 31 comprises at least one ball-and-socket link 40 as shown in FIG. 4 that has a male portion inserted into an adjacent female portion such that the semi-rigid portion may be repeatably bent to any predetermined angle and returned to a pre-bent orientation by a user while maintaining the structural integrity of the semi-rigid portion 31. In the configuration depicted in FIG. 3, the semi-rigid portion includes a protective sheath 37.

The ball-and-socket links 40, shown in FIG. 4, may be formed from various polymers. These particular polymers are usable in the medical device field because they are biocompatible. These polymers are also very heat resistant and exhibit reasonable structural strength and resilience. The ball-and-socket links 40 may also be molded, cast, milled, modified, or otherwise formed of other materials including certain metals.

Examples of biocompatible materials include, for example, various metals, polymers, or the like, such as polyetherimide (“PEI”). PEI is commercially available, for example, under the trademark Ultem 1000®. PEI materials are favorable in a flexible-shaft surgical instruments because materials such as PEI are approved for use in medical devices. Additional advantages are that these materials can be more easily formed to desired structures by varying processes such as machining or injection molding, than other bio-compatible materials such as certain metals. These materials are also non-conductive, have relatively high strength, are elastically expandable without fracturing or plastic deformation, have high wear resistance, and are rated for high temperature use, making them autoclavable. Rating for high temperature use is important so that the flexible-shaft surgical instrument may survive multiple sterilizations and be re-used a plurality of times without adversely affecting the structural integrity of the bendable portions.

PEI is particularly advantageous because it is also creep resistant. Creep is an inherent condition of certain plastics and polymers where the strength of the material is gradually lost over time if the material is repeatedly or consistently exposed to a loading or bending force. Loading and bending forces would be present in exemplary embodiments such as those described here including press fit ball-and-socket links. Other typical biocompatible polymers are less creep resistant and would thus have a shorter shelf life if used according to the exemplary embodiments. Because PEI is creep resistant, the shelf life may exceed that of typical biocompatible polymers if used according to the exemplary embodiments. When assembled, parts using PEI are under a load condition that exhibits stresses on the assembled parts which may tend to promote deformation from creeping. PEI is also non-reactive to most chemicals found in surgical procedures.

The ball-and-socket links 40 may be detachably connected to the outer tube 36 so that the semi-rigid portion 31 may be interchangeable with other configurations of semi-rigid portions. The ball-and-socket links 40 govern the bendability of the semi-rigid portion 31.

The degree of bend of the semi-rigid portion may be dependent upon the size or number of the ball-and-socket links 40, as well as the amount of friction that these links 40 experience between one another. The size of the ball-and-socket links 40 may also determine the amount of force that is required to bend the semi-rigid portion 31 to a desired bend radius, and the resistance of the device to move while in use, based, for example, on the amount of frictional surface area of adjacent links 40 at their point of connection.

Various arrangements of the number of links 40 as well as the size of the links 40 themselves govern the particular geometric configurations that the semi-rigid portion is capable of achieving. For instance, if there is only one ball-and-socket link 40, there is no bend radius in the semi-rigid portion 31 and the degree to which the semi-rigid portion 31 may be turned is limited to the longitudinal axis of the device. With two ball-and-socket links 40 the semi-rigid portion may be limitedly bent in a single plane. As the number of ball-and-socket links 40 increases, a particular bend radius may be customized to any radius such that multiple bends at varying angles may be achieved in multiple planes. Further, the degree to which particular portions of the semi-rigid portion 31 may be bent may be dependent upon the sizes of the ball-and-socket links 40.

In some applications, a surgeon, for instance, may wish to bend the semi-rigid portion 31 in a particular arrangement and hold that arrangement during use. In other applications, the surgeon may wish to have a more flexible feed as he manipulates the flexible-shaft surgical instrument into position. The flexible shaver blade 30, therefore, may be manipulated with minimal force.

The flexible shaver blade 30 may have a protective sheath 37 that covers at least the semi-rigid portion 31, but may cover the entire outer tube 36. The protective sheath 35 may be desirable to keep the ball-and-socket links 40 free from any debris that may inhibit their functionality or shorten their life expectancy. The sheath may also be used to provide hermeticity of the flexible shaver blade 30. The sheath may also be used as insulation over the proximal and distal portions of the outer tube 36 when incorporating electrosurgical components to the device.

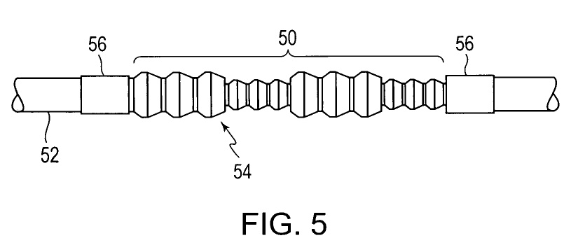

FIG. 5 illustrates a perspective view of a semi-rigid portion 50 of a flexible-shaft surgical instrument 52 that has ball-and-socket links 54 that vary in size and are interchangeable. The semi-rigid portion 50 has a collar 56 at each end that facilities the interchangeability of the semi-rigid portion 50 into and out of the flexible-shaft surgical instrument 52.

Various embodiments may include different sized ball-and-socket links 54 to account for varying bend radii, as well as varying holding strengths. The force at which the semi-rigid portion 50 may be bent is dependent upon the particular press fit between the male portion and adjacent female portion of the ball-and-socket links 54. The amount of friction between these two pieces may be equal or variable across a plurality of ball-and-socket links 54 that make up the semi-rigid portion 50, depending on the particular mating diameters of each male and adjacent female portion of the ball-and-socket links 54.

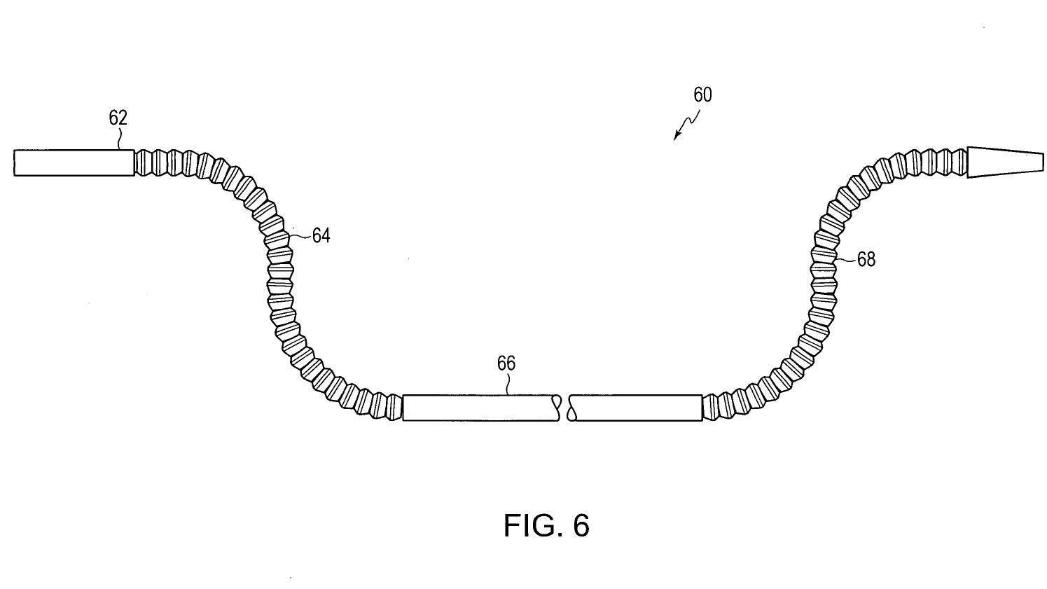

FIG. 6 illustrates a perspective view of a flexible-shaft surgical instrument 60 having a first rigid portion 62 connected to a first semi-rigid portion 64, a second rigid portion 66 that is connected to the first semi-rigid portion 62 and a second semi-rigid portion 68. The first and second semi-rigid portions 64 and 68 enable the first and second rigid portions 62 and 66 to be orientable in the same or varying planes.

It will be appreciated that the various of the above-disclosed and other features and functions, or alternatives thereof, may be desirably combined into many other different devices or applications. Also, various presently unforeseen or unanticipated alternatives, modifications, variations or improvements therein may be subsequently made by those skilled in the art which are also intended to be encompassed by the following claims.

What is claimed is:

1. A flexible-shaft surgical instrument, comprising:
at least one rigid tube portion;
at least one semi-rigid tube portion connected to the at least one rigid tube portion, the semi-rigid tube portion comprising a plurality of ball-and-socket links, each of the ball-and-socket links including a male portion and a female portion, the male portion being insertable into the female portion of an adjacent ball-and-socket link to form a press fit connection between the adjacent ball-and-socket links, the at least one semi-rigid tube portion being configured to be bent to a predetermined angle and returned to a pre-bent orientation repeatably and without plastic deformation; and
at least one surgical device,
wherein an internal diameter of the at least one rigid tube portion and the at least one semi-rigid tube portion accommodates the at least one surgical device, and access for the at least one surgical device to a target site in a patient's body is provided through the surgical instrument via at least one opening in or adjacent to a distal end of the surgical instrument.

2. The surgical instrument of claim 1, wherein the at least one semi-rigid tube portion is connected to a distal end of the at least one rigid tube portion.

3. The surgical instrument of claim 1, wherein the plurality of adjacent ball-and-socket links is of a number and configuration large enough such that the at least one semi-rigid portion may be bent between an angle of 0° and 275°.

4. The surgical instrument of claim 1, wherein the plurality of adjacent ball-and-socket links are substantially equal in size.

5. The surgical instrument of claim 1, wherein the plurality of adjacent ball-and-socket links vary in size.

6. The surgical instrument of claim 1, wherein the at least one rigid tube portion comprises at least two rigid tube portions.

7. The surgical instrument of claim 1, wherein the at least one semi-rigid tube portion comprises at least two semi-rigid tube portions,
wherein first and second of the at least two semi-rigid tube portions are configured to be orientated in the same or varying planes.

8. The surgical instrument of claim 1, wherein at least one portion of the female portion of at least one of the plurality of adjacent ball-and-socket links overlaps the male portion of the adjacent ball-and-socket link that is inserted into the female portion.

9. The surgical instrument of claim 1, further comprising a protective sheath covering at least the at least one semi-rigid portion.

10. The surgical instrument of claim 1, wherein the male portion of the at least one pair of adjacent ball-and-socket links may be popped into the adjacent female portion of the at least one ball-and-socket link.

11. The surgical instrument of claim 1, wherein a material for the plurality of ball-and-socket links provides a friction between the male portion of the at least one pair of adjacent ball-and-socket links and the female portion of the at least one adjacent ball-and-socket link so that the semi-rigid portion holds its predetermined bend angle.

12. The surgical instrument of claim 1, wherein the outer diameter of the male portion is 3-5% larger than the internal diameter of the adjacent female portion prior to assembly.

13. The surgical instrument of claim 1, wherein at least one pair of adjacent ball-and-socket links comprises a material that is non-conductive.

14. The surgical instrument of claim 1, wherein at least one pair of adjacent ball-and-socket links comprises a material that is temperature resistant.

15. The surgical instrument of claim 1, wherein at least one pair of adjacent ball-and-socket links comprises a material that is molded.

16. The surgical instrument of claim 1, wherein at least one pair of adjacent ball-and-socket links comprises a material that is biocompatible.

17. The surgical instrument of claim 1, wherein at least one pair of adjacent ball-and-socket links comprises a material that is not degraded by chemicals employed in a surgical procedure.

18. The surgical instrument of claim 1, wherein the at least one surgical device comprises at least one of a shaver, light, vacuum, suction lumen, camera, observation device, microdebrider, or electro-surgical probe.

19. The surgical instrument of claim 1, wherein at least one pair of adjacent ball-and-socket links is formed from a polymer material.

20. The surgical instrument of claim 19, wherein the polymer material is polyetherimide.
